(19) United States
(12) Patent Application Publication
(10) Pub. No.: US 2026/0335254 A1
(43) Pub. Date: Oct. 29, 2026

(54) PROTEIN CARRIER-BASED SYSTEM FOR TRANSDERMAL DELIVERY OF ACTIVE INGREDIENT TO HAIR FOLLICLE

(71) Applicant: SHANGHAI JIAO TONG UNIVERSITY, Shanghai (CN)

(72) Inventors: Xin JIN, Shanghai (CN); Jixiang LI, Shanghai (CN); Xinyuan ZHU, Shanghai (CN)

(21) Appl. No.: 19/703,514

(22) Filed: Jun. 10, 2026

Related U.S. Application Data
(63) Continuation-in-part of application No. PCT/CN2024/119335, filed on Sep. 18, 2024.

(30) Foreign Application Priority Data
Jun. 18, 2024 (CN) 202410787794.4

(51) Int. Cl. A61K 31/122; A61K 9/06; A61K 9/51; A61K 47/62; A61P 17/14

(52) U.S. Cl. CPC A61K 31/122; A61K 9/06; A61K 9/5161; A61K 47/62; A61P 17/14

(57) ABSTRACT

A protein carrier-based system for transdermal delivery of active ingredients to hair follicles is of a composite structure composed of small molecule active ingredients, bioactive protein and a polymer shell and has a size of 40-150 nm. The polymer shell has a thickness of 10-70 nm. The protein carrier-based system has a positive Zeta potential. The small molecule active ingredients are loaded in the bioactive protein, and the polymer shell coats the bioactive protein to form a protein nanogel. After the protein carrier-based system is applied to hair follicles or the skin surface, functional small molecules are transdermally delivered into the hair follicles and surrounding tissues thereof along with the nanogel, and then released in a controlled manner, thereby exerting the effects effectively.

### TECHNICAL FIELD

The present disclosure relates to the technical field of biomedicine and the technical field of composite materials for transdermal delivery, in particular to a protein carrier-based system for transdermal delivery of active ingredients to hair follicles.

### BACKGROUND

Hair is an important component of human appearance. Bad hair growth conditions such as abnormal hair loss and white hair not only weaken the protection of the body, but also significantly affect people's mental health. Hair-related diseases are caused by changes in the complex environment of hair follicles and their surrounding tissues, and therefore, research on delivery of active ingredients to hair follicles for the treatment of related diseases has attracted increasing attention.

The hair follicles are of a skin accessory structure formed by the invagination of the epidermis into the dermis and are of a relatively complete structure composed of multiple cell populations such as keratinocytes, melanocytes, and hair follicle mesenchymal stem cells. An upper segment of the hair follicle structure is a permanent portion, including the infundibulum and the isthmus; a lower segment of the hair follicle structure is a bulb, containing the hair papilla, which is a growth site for hair and hair follicles. The infundibulum at the upper ends of the hair follicles is of an open structure. During transdermal drug delivery, the unobstructed hair follicle openings are conducive to the direct penetration of drugs to bypass the physical barrier of the epidermis. However, in the scalp and facial regions, the total area of the hair follicle openings accounts for only 10% of the total skin area, and some hair follicles are blocked by shed keratinocytes and oil, preventing the drug penetration. A large number of immune cells including Langerhans cells surround the upper parts of the hair follicles. An immune barrier formed by the immune cells will recognize and remove drugs that penetrate into tissues around the hair follicles. In addition, in order to make the delivered drugs stay in or around the hair follicles, the size range of the drugs is also limited. Particles with too large a size (greater than 500 nm) will not be able to reach deep parts of the hair follicles smoothly, while particles with too small a size (less than 10 nm) will directly penetrate through the bottoms of the hair follicles into deep layers, with a residence time that is too short to exert the effect. Therefore, although there are some active drugs for hair follicles and hair problems, including retinoic acids, antioxidants, minoxidil, etc., how to efficiently deliver drugs to hair follicles transdermally and exert their effects is still a research difficulty and focus.

Currently, clinical or commercial technologies for promoting the delivery of active ingredients to hair follicles mainly adopt penetration enhancers. The use of commonly used penetration enhancers such as ethanol may affect the activity of some small molecule components, and the addition of the penetration enhancers is not conducive to the growth and development of skin and hair follicles, facing the risk of irritation and allergy. Microneedles belong to an emerging method for treating hair follicle diseases in recent years. The microneedles use mechanical external forces to create micro-pores in the skin around hair follicles to help drugs avoid the skin barrier, efficiently delivering the drugs to deep parts of the skin and then penetrating into the hair follicles. However, the frequent use of the microneedles will lead to local skin damage and induce inflammation, which in turn exerts an adverse effect on the growth and development of the hair follicles.

The inventors' prior patents CN202311704896.7, CN202410225688.7, and CN202410286148.X respectively relate to a tyrosinase-based nanotransdermal delivery system for treating vitiligo, a nanotransdermal delivery system for glaucocalyxin A for resisting skin inflammation, and a nanotransdermal delivery system for glaucocalyxin A for resisting skin inflammation. In view of the transdermal delivery environment of hair follicles, this patent selects the size of a nanosystem suitable for delivery in hair follicles, and optimizes and adjusts the hydrophilic and hydrophobic properties of a polymer shell material, and active proteins and active small molecules are preferred.

### SUMMARY

In order to overcome the defects of low delivery efficiency and uncontrollable effects on the growth and development of hair follicles of the existing drug delivery technology for hair follicle diseases, the present disclosure uses protein molecules as a nano template to prepare a protein nanogel by polymer wrapping, so as to construct a non-invasive and efficient system for transdermal delivery to hair follicles. By using the hydrophobicity inside the protein, active small molecule drugs with a hydrophobic structure can be loaded to achieve efficient delivery of active small molecules to hair follicles. The protein gel used in the transdermal delivery system has a special size. By controlling the loading amount of small molecules and the thickness of a polymer shell, the size of a nanogel carrier can be controlled, which is conducive to efficient penetration and retention. By customizing the surface properties of the polymer shell on an outer layer of the protein, the delivery system can effectively penetrate a physical barrier and an immune barrier of the skin, penetrate through the surrounding skin into the bottoms of the hair follicles, and then penetrate into the hair follicles or directly penetrate through the hair follicles to efficiently reach deep parts of the hair follicles, so as to exert physiological functions.

In addition, the protein nanogel is less irritating to the skin and hair follicles, and does not destroy the normal physiological functions of the hair follicles and skin during delivery. The protein nanogel can protect the stability of active ingredients, and effectively avoid the irritation of some active ingredients on the skin and hair follicles. The excellent penetration efficiency of the nanogel allows the active ingredients to efficiently reach the deep parts of the hair follicles and surrounding tissues, so as to exert the effects, so that the hair problems can be effectively improved by using lower concentrations of active ingredients, and the waste and irritation of high-concentration drugs can be effectively avoided. This loading method has universality for the active small molecules with the hydrophobic structure.

The present disclosure provides the following technical solutions:

Provided is a protein carrier-based system for transdermal delivery of active ingredients to hair follicles, wherein the system is of a composite structure composed of small molecule active ingredients, bioactive protein and a polymer shell and has a size of 40-150 nm, and the polymer shell has a thickness of 10-70 nm; the protein carrier-based system for transdermal delivery of active ingredients to hair follicles has a positive Zeta potential; and the small molecule active ingredients are loaded in the bioactive protein, and the polymer shell coats the bioactive protein. The small molecule active ingredients and the bioactive protein have beneficial active effects on the hair follicles.

The protein carrier-based system for transdermal delivery of active ingredients to hair follicles of the present disclosure may be simply referred to as a bioactive protein nanogel.

Further, the small molecule active ingredients bind to bioactive protein molecules by adsorption, hydrophilic and hydrophobic interactions, electrostatic interaction, hydrogen bonding, etc., and have a structure including one or more of the following characteristics: a positively charged group, a hydrophobic group, and a group capable of forming hydrogen bonds.

Further, the small molecule active ingredients can exert physiological functions for the hair follicle problems, and examples thereof include, but are not limited to, the following situations: for hormonal alopecia, the small molecule active ingredients include sulforaphane; for soft and fine hair with hair follicle dysplasia, the small molecule active ingredients include vitamin A; for the problem of oxidative stress in the hair follicles, the small molecule active ingredients include at least one of carotenoids, hesperidin and vitamin E; and for the problem of hair follicle inflammation, the small molecule active ingredients include at least one of resveratrol, lycopene, and astaxanthin.

Further, the bioactive protein has a size of 0.5-20 nm, preferably 3-10 nm. It is difficult for the bioactive protein molecules themselves to penetrate the skin barrier and enter the hair follicles. Even if a very small number of protein molecules enter the hair follicles, their sizes also restrict the transport capacity, making it difficult to reach the deep parts of the hair follicles to exert the effects. In addition, the microenvironment inside the hair follicles is not conducive to the long-term function of the protein, and even if the protein is delivered into the hair follicles, the protein is also inactivated in a short time. The poor solubility of most active small molecules first limits their individual applications. In addition, small molecules also have the problem of low delivery efficiency inside the hair follicles, and even if the small molecules enter the hair follicles, there remains a risk of inactivation or direct penetration through the hair follicles without retention to exert their effects. In the present disclosure, the size of the delivery system is increased and the surface potential is changed to be positive by polymer coating modification on the protein. After the size increase, the overall delivery of the nanogel in the hair follicles conforms to the ratchet effect, which is beneficial to the delivery of active ingredients to living cells in the deep parts of the hair follicles to exert the effects, instead of staying on the surfaces of pores or directly passing through the hair follicles. In addition, the polymer shell protects the activity of the protein and small molecules inside, and the small molecules are slowly released from the protein, which is conducive to the long-term therapeutic effect of the protein nanogel in the deep parts of the hair follicles.

Further, examples of exerting physiological functions for the hair follicle problems include, but are not limited to, the following situations: for the problem of hair hypopigmentation, the bioactive protein includes tyrosinase; for the problem of oxidative stress in the hair follicles, the bioactive protein includes at least one of horseradish peroxidase, peroxidase, and superoxide dismutase; for the problem of hair follicle inflammation, the bioactive protein includes at least one of lysozyme, papain, and bromelain; for the problem of excessive oil secretion, the bioactive protein includes lipase; and for the problem of hair loss caused by excessive secretion of male hormones, the bioactive protein includes 3β-hydroxysteroid dehydrogenase.

Further, the protein carrier-based system for transdermal delivery of active ingredients to hair follicles has a Zeta potential of +3 mV to +7 mV. The positive surface potential facilitates the interaction of the nanogel with negatively charged lipid components in pores and the skin barrier, weakening the barrier effect and facilitating the delivery of the nanogel.

Further, the polymer shell is obtained by polymerization of monomers, and a coating mode includes in-situ polymerization or the polymer shell coats the surface of the bioactive protein nanogel by electrostatic attraction; or the polymer shell is a natural polymer and coats the surface of the bioactive protein nanogel by hydrogen bonding or electrostatic interaction.

Further, the polymer shell is obtained by polymerization of monomers. The monomers are selected from vinylpyrrolidone, acrylamide, N-propenylsuccinimide, 2-methacryloyloxyethylphosphocholine, [2-(methacryloyloxy)ethyl]dimethyl-(3-sulfopropyl)ammonium hydroxide, and acrylate when the polymer shell coats the surface of the bioactive protein nanogel by in-situ polymerization; the acrylate is at least one selected from methyl (meth)acrylate, ethyl (meth)acrylate, hydroxyethyl (meth)acrylate, propyl (meth)acrylate, and butyl (meth)acrylate; when the polymer shell coats the surface of the protein by electrostatic interaction, the polymer shell is a positively charged polymer, i.e., at least one of poly(acrylamine hydrochloride), poly(L-lysine), polyethyleneimine, poly(L-histidine), poly(N,N-dimethylaminoethyl methacrylate) and polymethacrylamidopropyltrimethylammonium chloride; or the polymer shell is a natural polymer including at least one of polysaccharide and chitosan.

Further, a mass ratio of the small molecule active ingredients to the bioactive protein is 1:1-10, preferably 1:2-5.

A second object of the present disclosure is to provide a preparation method for the protein carrier-based system for transdermal delivery of active ingredients to hair follicles, wherein the method is one of the following methods:
when a polymer is obtained by polymerization of monomers, the method adopts in-situ polymerization, and includes the steps of:
(S1) adding a solution of a surface modification monomer to a bioactive protein solution to obtain a surface-modified protein solution;
(S2) adding a small molecule active ingredient solution to the surface-modified protein solution obtained in the step (S1), and loading small molecule active ingredients in bioactive protein; and
(S3) adding a functional monomer, a positively charged monomer, a crosslinking agent and an initiator, and initiating in-situ polymerization to obtain the protein carrier-based system for transdermal delivery of active ingredients to hair follicles;
or,
when a shell is obtained via self-assembly of a positively or negatively charged polymer on the surface of protein, the method includes the step of:
(T1) adding a bioactive protein solution to a positively or negatively charged polymer solution under the condition of a water bath of 50-70 °C with stirring, performing stirring, adding a small molecule active ingredient solution under the condition of an ice water bath, adding a salt solution, performing stirring, and performing ultrafiltration centrifugation to obtain the protein carrier-based system for transdermal delivery of active ingredients to hair follicles.

Further, in the step (S1), the bioactive protein solution has a concentration of 1-5 mg/mL and is a buffer solution having a pH of 7.2-7.6, such as a phosphate buffer solution; the surface modification monomer is at least one selected from N-propenylsuccinimide, N-(3-aminopropyl)methacrylamide hydrochloride, and maleimide, and the function of the surface modification monomer is to modify the monomer on the surface of protein through electrostatic adsorption, chemical bonding and other interactions; the solution of the surface modification monomer has a concentration of 1-5 wt %, and a solvent the solution of the surface modification monomer is at least one of dimethyl sulfoxide, ethanol, propanol, and ethylene glycol; and a mass ratio of a bioactive protein to the surface modification monomer is 1:0.05-0.1, such as 1:0.075.

Further, in the step (S2), the small molecule active ingredient solution has a concentration of 0.5-5 mg/mL, and a solvent of the small molecule active ingredient solution is at least one of water, methanol, ethanol and ethylene glycol; and after adding the small molecule active ingredient solution, stirring is performed for 10-60 min under the stirring condition of 300-500 r/min.

Further, in the step (S3), the functional monomer is at least one selected from vinylpyrrolidone, acrylamide, 2-methacryloyloxyethylphosphocholine, and 2-hydroxyethyl acrylate; the positively charged monomer is at least one selected from ethyl 2-(dimethylamino)methacrylate, (3-acrylamidopropyl)trimethylammonium chloride hydrochloride, and N-(3-aminopropyl)methacrylamide hydrochloride; the crosslinking agent is at least one selected from N,N′-methylenebisacrylamide, glycerol dimethacrylate, and glycerol 1,3-diglycerolate diacrylate; the initiator is a water-soluble initiator, and is specifically at least one selected from ammonium persulfate, sodium persulfate and potassium persulfate; and 2-5 mg of the initiator is added per milligram of the bioactive protein.

Further, in the step (S3), a molar ratio of the bioactive protein to the functional monomer to the positively charged monomer is 1:11000-52000:1000-6000, and the amount of the crosslinking agent used is 5-10% of a molar amount of the functional monomer.

Further, in the step (S3), a co-initiator such as sodium bisulfite or tetramethylethylenediamine is further added. 5-10 mg of the co-initiator is added per milligram of the protein.

A third object of the present disclosure is to provide use of the protein carrier-based system for transdermal delivery of active ingredient to hair follicles in the manufacture of a medicament for the prevention or treatment of diseases including bacterial infection of hair follicles, seborrheic folliculitis, seborrheic alopecia, androgenetic alopecia and the like.

Compared with the prior art, the present disclosure achieves the following beneficial effects:
1. In the present disclosure, the protein is modified with compounds with polymerizable double bonds, after the small molecule active ingredients are loaded, the polymerization reaction is initiated in situ on the surface of the protein to coat the surface of the protein with a polymer protective layer which is bio-friendly and has no or little irritation to the skin. During the transdermal delivery process, by regulating and controlling the surface properties and thickness of the polymer protective layer, the penetration ability of the protein nanogel in the hair follicles and surrounding skin tissues thereof can be effectively regulated and controlled, so that the nanogel can efficiently penetrate the skin as a whole to reach the positions where the active ingredients are required to exert their effects. The polymer layer of the present disclosure not only provides effective protection for the bioactive protein nanogel inside, but also does not affect the exertion of the physiological activity of the bioactive protein nanogel and bioactive small molecules loaded in the bioactive protein nanogel. The hair follicle problems are solved in a targeted manner. The defect of poor delivery efficiency of small molecules is significantly improved.
2. For common active ingredients that have certain irritation to the skin and hair follicles, after they are loaded in the protein nanogel, the nanogel not only promotes their penetration depth, but also reduces their irritation to the skin, making them suitable for a wider range of people.
3. Raw materials used in the present disclosure are all bio-friendly and can be used as reagents for drugs, so they can be used in transdermal delivery systems for ingredients beneficial to the human body such as drugs. The preparation method for the transdermal delivery system of the present disclosure is simple, and the transdermal delivery system has high yield, and can be industrially produced on a large scale.

### BRIEF DESCRIPTION OF DRAWING(S)

FIG. 1 is an SEM image of polyethylene glycol-3β-hydroxysteroid dehydrogenase nanogel (PEG-nHSD@Res) in Example 1.

FIG. 2 is an SEM image of chitosan-superoxide dismutase nanogel (CS-nSOD@Ast) in Example 2.

FIG. 3 is an SEM image of polyvinylpyrrolidone-bromelain nanogel (PVP-nBM@VA) in Example 3.

FIG. 4 shows hair follicle growth and development over a 20-day administration period of PVP-nBM@VA in Example 3.

### DETAILED DESCRIPTION

Hereinafter, the present disclosure will be described with reference to specific examples, but the present disclosure is not limited thereto.

The experimental methods used in the following examples are conventional methods unless otherwise specified. The reagents, materials and the like used in the following examples are commercially available unless otherwise specified.

The concentration of a protein nanogel solution in the examples of the present disclosure is in terms of a mass of protein.

### Example 1

To 2 mL of a 3β-hydroxysteroid dehydrogenase (HSD) solution with a concentration of 1 mg/mL, 0.15 mg of N-propenylsuccinimide was added, wherein N-propenylsuccinimide was dissolved in dimethyl sulfoxide. Well mixing was performed, and a reaction was carried out for 2 h to obtain 3β-hydroxysteroid dehydrogenase modified with polymerizable double bonds on the surface (aHSD).

To 2 mL of a 1 mg/mL solution of 3β-hydroxysteroid dehydrogenase modified with double bonds on the surface, 0.45 mL of a methanol solution of resveratrol with a concentration of 0.5 mg/mL was added dropwise at 350 r/min while stirring. Well mixing was performed to obtain resveratrol-loaded 3β-hydroxysteroid dehydrogenase (aHSD@Res).

aHSD@Res, polyethylene glycol methyl ether acrylate, aminopropylmethacrylamide hydrochloride and N,N′-methylenebisacrylamide were mixed at a molar ratio of 1:8000:1000:800, the protein concentration was diluted to 1 mg/mL with phosphate buffer having a pH of 7.4, then an initiator was then added according to 3.8 mg of ammonium persulfate and 7.6 mg of tetramethylethylenediamine per milligram of protein, and in-situ free radical polymerization was initiated at room temperature to obtain resveratrol-loaded polyethylene glycol-3β-hydroxysteroid dehydrogenase nanogel (PEG-nHSD@Res) after reacting for 4 h.

The obtained compound solution was subjected to ultrafiltration treatment in an ultrafiltration centrifuge tube having a molecular weight cutoff of 3 kDa to obtain a purified high-concentration mother liquor having a concentration of 5.7 mg/mL, and the mother liquor was stored under refrigeration.

### Example 2 Preparation of Chitosan-Superoxide Dismutase Nanogel

6 mL of a chitosan solution with a concentration of 1 mg/mL was taken and sonicated at a power of 70 W for 10 minutes and allowed to stand to avoid excessive heat generation. In a water bath at 60 °C, a superoxide dismutase solution was added, and the concentration of superoxide dismutase in the solution was adjusted to 1 mg/mL, and a reaction was carried out for 5 min. To the mixed solution, an astaxanthin acetone solution with a concentration of 0.5 mg/mL was added dropwise at 300 r/min in a water bath of 60 °C water bath, and stirring was continued to be performed for 5 min. Finally, 1 mL of a sodium tripolyphosphate solution with a concentration of 1 mg/mL was added dropwise under the condition of an ice-water bath at 300 r/min, and a reaction was carried out for 30 min to obtain astaxanthin-loaded chitosan-superoxide dismutase nanogel (CS-nSOD@Ast).

The obtained compound solution was subjected to ultrafiltration treatment in an ultrafiltration centrifuge tube having a molecular weight cutoff of 3 kDa to obtain a purified high-concentration mother liquor having a concentration of 6.1 mg/mL, and the mother liquor was stored under refrigeration.

Characterization of the size of the nanogel by dynamic light scattering (DLS): a protein nanogel solution with a concentration of 1 mg/mL was added to a sample cell, and the particle size and Zeta potential of protein nanogel in the system were tested at room temperature by using a Malvern Nano Zs90 nanoparticle size potentiometer. The results are statistically shown in Table 1.

### Example 3 Preparation of Polyvinylpyrrolidone-Bromelain Nanocapsules

To 4 mL of a 0.5 mg/mL bromelain (BM) solution, 0.5 mg of N-propenylsuccinimide was added, wherein N-propenylsuccinimide was dissolved in dimethyl sulfoxide. Well mixing was performed, and a reaction was carried out for 4 h to obtain bromelain modified with polymerizable double bonds on the surface (aBM).

To 4 mL of a 0.5 mg/mL solution of bromelain modified with double bonds on the surface, 0.6 mL of an ethanol solution of vitamin A with a concentration of 0.3 mg/mL was added dropwise at 300 r/min while stirring. Well mixing was performed to obtain vitamin A-loaded bromelain (aBM@VA).

aBM@VA, N-vinyl-2-pyrrolidone, aminopropylmethacrylamide hydrochloride and N,N′-methylenebisacrylamide were mixed at a molar ratio of 1:6000:1000:600, the protein concentration was dilutes to 1 mg/mL with phosphate buffer having a pH of 7.4, an initiator was then added according to 3.8 mg of ammonium persulfate and 7.6 mg of tetramethylethylenediamine per milligram of protein, and in-situ free radical polymerization was initiated at room temperature to obtain vitamin A-loaded polyvinylpyrrolidone-bromelain nanogel (PVP-nBM@VA).

The obtained compound solution was subjected to ultrafiltration treatment in an ultrafiltration centrifuge tube having a molecular weight cutoff of 3 kDa to obtain a purified high-concentration mother liquor having a concentration of 6.1 mg/mL, and the mother liquor was stored under refrigeration.

TABLE 1
Particle size and Zeta potential of protein nanogel

| | Particle size distribution | Zeta potential |
|---|---|---|
| Sample name | center (nm) | (mV) |
| Native 3β-hydroxysteroid | 6 | −5.9 ± 2.0 |
| dehydrogenase | | |
| PEG-nHSD@Res | 68 | +3.8 ± 1.7 |
| Native superoxide | 5 | −7.1 ± 3.8 |
| dismutase | | |
| CS-nSOD@Ast | 117 | +6.3 ± 2.1 |
| Native bromelain | 9 | −5.4 ± 1.7 |
| PVP-nBM@VA | 60 | 4.9 ± 1.5 |

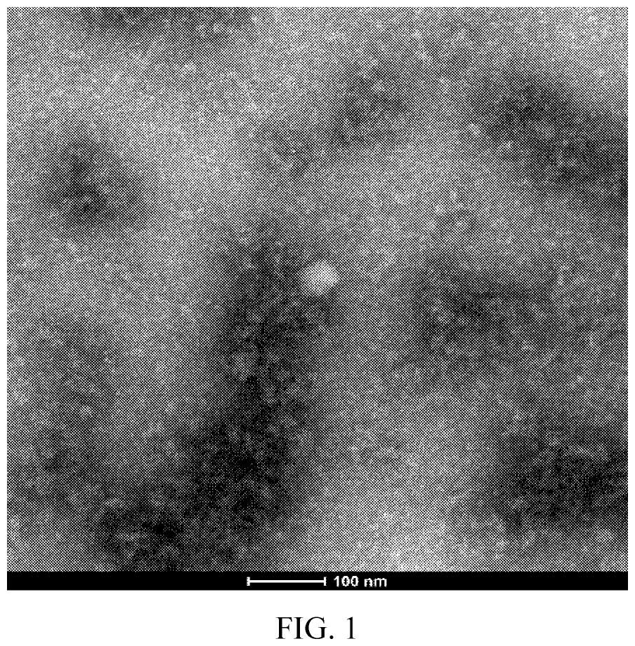

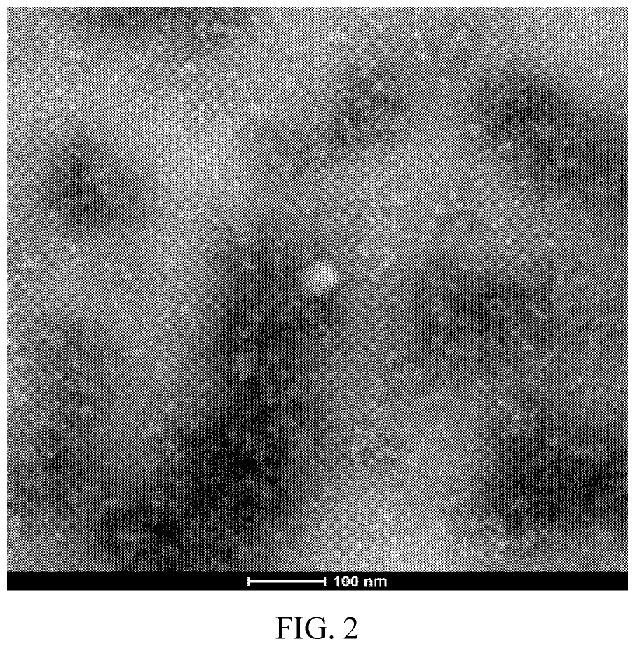

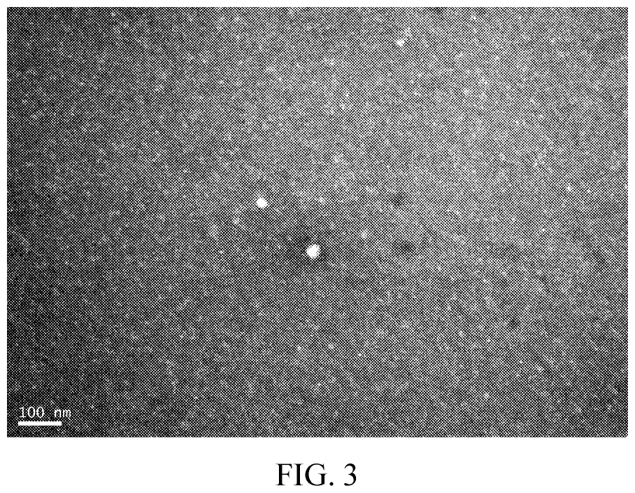

Characterization of the morphology of the nanogel by transmission electron microscopy (TEM): the morphology and dispersion of the polyethylene glycol-3β-hydroxysteroid dehydrogenase nanogel (PEG-nHSD@Res) in Example 1 were observed under a transmission electron microscope, and the results are shown in FIG. 1. The morphology and dispersion of the chitosan-superoxide dismutase nanogel (CS-nSOD@Ast) in Example 2 were observed under a transmission electron microscope, and the results are shown in FIG. 2. The morphology and dispersion of the polyvinylpyrrolidone-bromelain nanogel (PVP-nBM@VA) in Example 3 were observed under a transmission electron microscope, and the results are shown in FIG. 3.

### Application Example 1 3β-Hydroxysteroid Dehydrogenase Activity Test

Test of the activity of 3β-hydroxysteroid dehydrogenase in the polyethylene glycol-3β-hydroxysteroid dehydrogenase nanogel (PEG-nHSD@Res) in Example 1: a native 3β-hydroxysteroid dehydrogenase solution and a solution of PEG-nHSD@Res in Example 1 were taken, the concentration was adjusted to 1 mg/mL, and their activity were determined by using an ELISA kit, and the results are shown in Table 2.

TABLE 2
Relative activity of HSD

| Sample name | Relative activity (%) |
|---|---|
| Native 3β-hydroxysteroid dehydrogenase | 100.0 ± 6.0 |
| PEG-nHSD@Res | 93.7 ± 2.9 |

PEG-nHSD@Res prepared in Example 1 also retained about 93.7% of the activity of the protein, indicating that the loss of enzyme activity caused by the modification of 3β-hydroxysteroid dehydrogenase and the preparation of the polymer shell by the method of the present disclosure was not significant and was within an acceptable range.

### Application Example 2 Superoxide Dismutase Activity Test

The activity of superoxide dismutase was determined by an ultraviolet spectrophotometer. 3 mL of 50 mmol/mL Tris-HCl buffer with a pH of 8 (containing 1 mmol/mL EDTA) was added to a reference cell, 2.4 mL of 50 mmol/mL Tris-HCl buffer with a pH of 8 (containing 1 mmol/mL EDTA), 0.3 mL of 0.2 mmol/mL pyrogallol, and 0.3 mL of native superoxide dismutase or enzyme nanogel solution were added to a sample cell, the reference cell and the sample cell were placed into the ultraviolet spectrophotometer, the change in absorbance of the sample cell at 420 nm in the first 10 minutes was recorded, and a slope of a fitting straight line was used to represent the relative activity of superoxide dismutase. The results are shown in Table 3.

TABLE 3
Relative activity of SOD

| Sample name | Relative activity (%) |
|---|---|
| Native superoxide dismutase | 100.0 ± 5.5 |
| CS-nSOD@Ast | 85.7 ± 4.6 |

It was indicated that CS-nSOD@Ast prepared in Example 2 also retained 85.7% of the activity, indicating that the loss of enzyme activity caused by the modification of superoxide dismutase and the preparation of the polymer shell was within an acceptable range.

### Application Example 3 Bromelain Activity Test

The activity of bromelain was determined by an ultraviolet spectrophotometer. 1 mL of a native bromelain solution or an enzyme nanogel solution was mixed with 3 mL of a 0.6 wt % casein solution, stirring was performed for 10 minutes, and a reaction was carried out. Then 3 mL of trichloroacetic acid was added to precipitate the protein, and the resulting reaction solution was allowed to stand for 10 min to terminate the reaction. The mixed solution was centrifuged at 4000 rpm for 3 min, and a supernatant was taken. 1 mL of the supernatant was mixed with 5 mL of a sodium carbonate solution with a concentration of 0.4 mol/L, and 1 mL of a folin-phenol reagent was added for color development. By using a system containing 1 mL of phosphate buffer, 5 mL of a sodium carbonate solution with a concentration of 0.4 mol/L and 1 mL of a folin-phenol reagent as a reference, an absorbance value A of a system containing the reaction supernatant was measured at a wavelength of 700 nm as the relative activity.

TABLE 4
Relative activity of BM

| Sample name | Relative activity (%) |
|---|---|
| Native bromelain | 100.0 ± 3.9 |
| PVP-nBM@VA | 87.3 ± 1.9 |

It was indicated that PVP-nBM@VA prepared in Example 3 also retained 87.3% of the activity, indicating that the loss of enzyme activity caused by the modification of bromelain and the preparation of the polymer shell was within an acceptable range.

### Application Example 4 Sustained Release of Active Ingredients

2 mL of the protein nanogel prepared in Examples 1-3 at a concentration of 5 mg/mL was placed into a dialysis bag having a molecular weight cutoff of 3000, and the dialysis bag was immersed in phosphate buffer with a pH of 6, and the entire system was shaken at 37 °C. The sustained release time was 48 h. Within 48 h, samples were taken at regular intervals, and the concentration of loaded small molecule drugs was measured under an ultraviolet spectrophotometer, and the sustained release of small molecule drugs by a composite system was calculated. The results are statistically shown in Table 4. Functional small molecules are slowly released from the protein nanogel within 24 hours, which is beneficial for the transdermal system to release the functional small molecules and exert their effects after reaching deep parts of hair follicles and surrounding tissues thereof.

TABLE 5
Release of small molecule drugs from the nanogel

| | Cumulative release rate (%) | | |
|---|---|---|---|
| Time (h) | PEG-nHSD@Res | CS-nSOD@Ast | PVP-nBM@VA |
| 0.25 | 6.3 ± 2.7 | 4.4 ± 1.1 | 7.1 ± 3.9 |
| 0.5 | 18.5 ± 3.6 | 17.9 ± 3.4 | 17.9 ± 2.1 |
| 1 | 29.7 ± 1.1 | 28.1 ± 5.5 | 30.3 ± 4.6 |
| 2 | 41.6 ± 6.2 | 35.9 ± 4.2 | 42.1 ± 5.4 |
| 4 | 62.9 ± 4.0 | 70.8 ± 9.3 | 71.7 ± 6.6 |
| 8 | 79.8 ± 3.3 | 77.9 ± 2.3 | 82.0 ± 5.1 |
| 24 | 80.2 ± 1.5 | 79.6 ± 1.8 | 83.4 ± 4.9 |

### Application Example 5 Validation of the Efficacy of Small Molecule Active Ingredients

Determination of the anti-inflammatory ability of the polyethylene glycol-3β-hydroxysteroid dehydrogenase nanogel (PEG-nHSD@Res): the NO concentration in inflammatory cells was detected by using a Griess kit. HaCaT cells (1*10⁵ cells/well) were inoculated in a 24-well plate. After 12 h, when the cells were completely adhered to the wall, the original medium was sucked away. 100 μL of a serum-free medium was added to all groups, 10 μL of a serum-free medium was added to a negative control group, 10 μL of PBS buffer was added to a positive control group, 10 μL of a PEG-nHSD@Res nanogel solution with a concentration of 0.01 mg/mL was added to a low-concentration experimental group, 10 μL of a PEG-nHSD@Res nanogel solution with a concentration of 0.02 mg/mL was added to a medium-concentration experimental group, and 10 μL of a PEG-nHSD@Res nanogel solution with a concentration of 0.04 mg/mL was added to a high-concentration experimental group. After co-incubation for 2 h, the original medium was sucked away from all groups, 110 μL of a fresh medium was added to the negative control group, and 100 μL of a fresh medium and 10 μL of interleukin-10 at a concentration of 10 ng/mL were added to the remaining groups. After co-incubation for 24 h, the determination was carried out according to the instructions of the Griess kit. The results are statistically shown in Table 5.

TABLE 6
Improvement of the intracellular anti-inflammatory ability
of PEG-nHSD@Res with the increase of concentration

| | | Positive | PEG- | PEG- | PEG- |
|---|---|---|---|---|---|
| | Negative | control | nHSD@Res | nHSD@Res | nHSD@Res |
| Experimental | control | group | with low | with medium | with high |
| group | group | (PBS) | concentration | concentration | concentration |
| NO | 23.0 ± 3.5 | 76.3 ± 8.0 | 66.5 ± 7.7 | 60.3 ± 5.1 | 42.6 ± 6.9 |
| concentration | | | | | |
| (μM) | | | | | |

The results show that the PEG-nHSD@Res nanogel has good anti-inflammatory ability, and this ability increases with the increase of concentration.

### Application Example 6 Animal Experiment

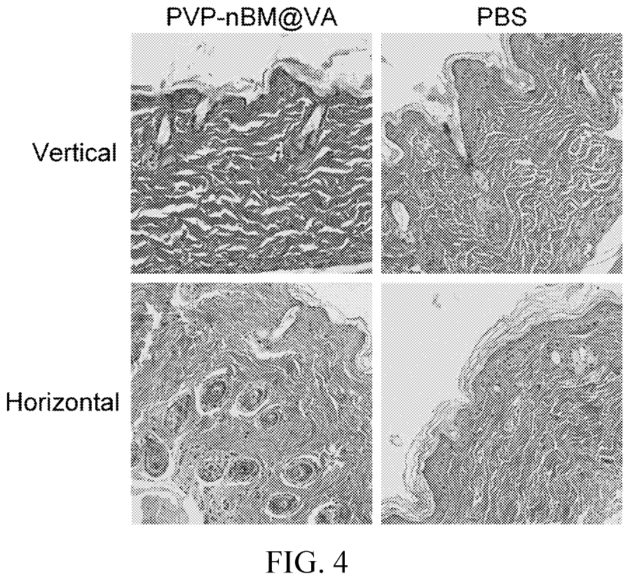

Determination of the ability of the polyvinylpyrrolidone-bromelain nanogel (PVP-nBM@VA) prepared in Example 3 to promote the growth and development of hair follicles: the effect of PVP-nBM@VA on the growth and development of hair follicles was investigated by using a Balb/c mouse model. 12 female mice were randomly divided into two groups, and the backs were subjected to unified hair removal treatment with an electric shaver, taking care not to damage native hair follicles. Then, 200 μL of a nanogel solution (an experimental group) or PBS buffer (a control group) were applied to the hair removal areas every two days, wherein the experimental group was given 300 unit/mL of PVP-nBM@VA, and the control group was given a buffer solution with a concentration of 20 mM and a pH of 6, and PVP-nBM@VA and the buffer solution were added dropwise to the hair removal areas and evenly spread. After 20 consecutive days of administration, skin on the back of the mice was taken for sectioning to observe the growth of hair follicles. The results are shown in FIG. 4, and the statistical results of hair follicle growth are shown in Table 7.

TABLE 7
Statistics on the length and number of hair follicles

| | Average length of hair | Average number of hair |
|---|---|---|
| Experimental group | follicles (mm) | follicles (pcs/mm²) |
| Control group (PBS) | 0.144 ± 0.041 | 27.8 ± 5.5 |
| PVP-nBM@VA | 0.279 ± 0.083 | 42.3 ± 9.7 |

The results showed that the length and average number of hair follicles in the mice administered with PVP-nBM@VA were significantly better than those in the control group during the 20-day administration period, indicating that PVP-nBM@VA promoted the increase of the number and length of the hair follicles.

1. A protein carrier-based system for transdermal delivery of active ingredients to hair follicles, wherein the system is of a composite structure composed of small molecule active ingredients, bioactive protein and a polymer shell and has a size of 40-150 nm, and the polymer shell has a thickness of 10-70 nm; the protein carrier-based system for transdermal delivery of active ingredients to hair follicles has a positive Zeta potential; and the small molecule active ingredients are loaded in the bioactive protein, and the polymer shell coats the bioactive protein.

2. The protein carrier-based system for transdermal delivery of active ingredients to hair follicles according to claim 1, wherein the small molecule active ingredients bind to bioactive protein molecules by adsorption, hydrophilic and hydrophobic interactions, electrostatic interaction, and hydrogen bonding, and have a structure comprising one or more of the following characteristics: a positively charged group, a hydrophobic group, and a group capable of forming hydrogen bonds.

3. The protein carrier-based system for transdermal delivery of active ingredients to hair follicles according to claim 1, wherein the small molecule active ingredients comprise at least one of sulforaphane, carotenoids, hesperidin, vitamin E, resveratrol, lycopene, and astaxanthin.

4. The protein carrier-based system for transdermal delivery of active ingredients to hair follicles according to claim 1, wherein the bioactive protein has a size of 0.5-20 nm, preferably 3-10 nm.

5. The protein carrier-based system for transdermal delivery of active ingredients to hair follicles according to claim 4, wherein the bioactive protein comprises at least one of tyrosinase, horseradish peroxidase, peroxidase, superoxide dismutase, lysozyme, papain, bromelain, lipase, and 3β-hydroxysteroid dehydrogenase.

6. The protein carrier-based system for transdermal delivery of active ingredients to hair follicles according to claim 1, having a Zeta potential of +3 mV to +7 mV.

7. The protein carrier-based system for transdermal delivery of active ingredients to hair follicles according to claim 1, wherein the polymer shell is obtained by polymerization of monomers, and a coating mode comprises in-situ polymerization or the polymer shell coats the surface of a bioactive protein nanogel by electrostatic attraction; or the polymer shell is a positively or negatively charged polymer, and coats the surface of the bioactive protein nanogel by hydrogen bonding or electrostatic interaction.

8. The protein carrier-based system for transdermal delivery of active ingredients to hair follicles according to claim 7, wherein the polymer shell is obtained by polymerization of monomers, the monomers are selected from vinylpyrrolidone, acrylamide, N-propenylsuccinimide, 2-methacryloyloxyethylphosphocholine, [2-(methacryloyloxy)ethyl]dimethyl-(3-sulfopropyl)ammonium hydroxide, and acrylate when the polymer shell coats the surface of the bioactive protein nanogel by in-situ polymerization; the acrylate is at least one selected from methyl (meth)acrylate, ethyl (meth)acrylate, hydroxyethyl (meth)acrylate, propyl (meth)acrylate, and butyl (meth)acrylate; when the polymer shell coats the surface of the protein by electrostatic interaction, the polymer shell is the positively charged polymer, i.e., at least one of poly(acrylamine hydrochloride), poly(L-lysine), polyethyleneimine, poly(L-histidine), poly(N,N-dimethylaminoethyl methacrylate) and polymethacrylamidopropyltrimethylammonium chloride; or the polymer shell is a natural polymer comprising at least one of polysaccharide and chitosan.

9. The protein carrier-based system for transdermal delivery of active ingredients to hair follicles according to claim 1, wherein a mass ratio of the small molecule active ingredients to the bioactive protein is 1:1-10, preferably 1:2-5.

10. A preparation method for the protein carrier-based system for transdermal delivery of active ingredients to hair follicles according to claim 1, being one of the following methods:
when a shell material is obtained by polymerization of monomers, the method adopts in-situ polymerization, and comprises the steps of:
(S1) adding a solution of a surface modification monomer to a bioactive protein solution to obtain a surface-modified protein solution;
(S2) adding a small molecule active ingredient solution to the surface-modified protein solution obtained in the step (S1), and loading small molecule active ingredients in bioactive protein; and
(S3) adding a functional monomer, a positively charged monomer, a crosslinking agent and an initiator, and initiating in-situ polymerization to obtain the protein carrier-based system for transdermal delivery of active ingredients to hair follicles;
when a shell is obtained via self-assembly of a positively or negatively charged polymer on the surface of protein, the method comprises the step of:
(T1) adding a bioactive protein solution to a positively or negatively charged polymer solution under the condition of a water bath of 50-70 °C with stirring, performing stirring, adding a small molecule active ingredient solution under the condition of an ice water bath, adding a salt solution, performing stirring, and performing ultrafiltration centrifugation to obtain the protein carrier-based system for transdermal delivery of active ingredients to hair follicles.

11. The preparation method according to claim 10, wherein in the step (S1), the bioactive protein solution has a concentration of 1-5 mg/mL and is a buffer solution having a pH of 7.2-7.6; the surface modification monomer is at least one selected from N-propenylsuccinimide, N-(3-aminopropyl)methacrylamide hydrochloride, and maleimide; the solution of the surface modification monomer has a concentration of 1-5 wt %, and a mass ratio of a bioactive protein nanogel to the surface modification monomer is 1:0.05-0.1; and/or
in the step (S2), the small molecule active ingredient solution has a concentration of 0.5-5 mg/mL, and a solvent of the small molecule active ingredient solution is at least one of water, methanol, ethanol and ethylene glycol; and/or
in the step (S3), the functional monomer is at least one selected from vinylpyrrolidone, acrylamide, 2-methacryloyloxyethylphosphocholine, and 2-hydroxyethyl acrylate; the positively charged monomer is at least one selected from ethyl 2-(dimethylamino)methacrylate, (3-acrylamidopropyl)trimethylammonium chloride hydrochloride, and N-(3-aminopropyl)methacrylamide hydrochloride; the crosslinking agent is at least one selected from N,N′-methylenebisacrylamide, glycerol dimethacrylate, and glycerol 1,3-diglycerolate diacrylate; the initiator is a water-soluble initiator, and is specifically at least one selected from ammonium persulfate, sodium persulfate and potassium persulfate; and 2-5 mg of the initiator is added per milligram of the protein.

12. The preparation method according to claim 10, wherein in the step (S3), a molar ratio of the bioactive protein to the functional monomer to the positively charged monomer is 1:11000-52000:1000-6000, and the amount of the crosslinking agent used is 5-10% of a molar amount of the functional monomer.

13. Use of the protein carrier-based system for transdermal delivery of active ingredients to hair follicles according to claim 1 in the manufacture of a medicament for the prevention or treatment of bacterial infection of hair follicles, seborrheic folliculitis, seborrheic alopecia, and androgenetic alopecia.
